(19) United States
(12) Patent Application Publication
(10) Pub. No.: US 2018/0030352 A1
(43) Pub. Date: Feb. 1, 2018

(54) REACTIVE PERPENDICULAR ALIGNED MATERIAL, LIQUID CRYSTAL DISPLAY PANEL, AND METHOD OF LIQUID CRYSTAL ALIGNMENT

(71) Applicant: Shenzhen China Star Optoelectronics Technology Co., Ltd., Shenzhen City (CN)

(72) Inventors: Song Lan, Shenzhen City (CN); Xiaolong Ma, Shenzhen City (CN); Yungjui Lee, Shenzhen City (CN)

(21) Appl. No.: 14/914,640

(22) PCT Filed: Dec. 24, 2015
(86) PCT No.: PCT/CN2015/098621

§ 371 Date: Feb. 26, 2016

(30) Foreign Application Priority Data
Nov. 16, 2015 (CN) 201510785878.5

(51) Int. Cl. C09K 19/56; G02F 1/1337; C08F 222/14; C07C 57/42; C07C 59/64

(52) U.S. Cl. CPC C09K 19/56; C07C 57/42; C07C 59/64; C08F 222/14; G02F 1/133719; G02F 1/133788; G02F 2001/133742

(57) ABSTRACT

The disclosure provides a reactive perpendicular aligned material, a liquid crystal display panel, and a method for aligning liquid crystals, a general structural formula of the reactive perpendicular aligned material is A-Z—R, A is —CH═CH—COOH; Z is

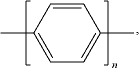

where n≧1; R represents a linear chained or a branched chained alkyl group with 5˜20 C atoms, a CH₂ group in the alkyl group is substituted by a phenyl group, a naphthenic base, —CONH—, —COO—, —O—CO—, —S—, —CO— or —CH═CH—, or a F atom or a Cl atom substitutes for a H atom in the alkyl group. A head group A plays a role in anchoring, it can be anchored on a surface of a substrate utilizing the —COOH group by means of physical reaction, meanwhile, it can strengthen the ability of anchoring liquid crystal molecules by the reaction of a —CH═CH— group and a reactive monomer

### FIELD OF THE DISCLOSURE

The present disclosure relates to a display technology field, and more particularly to a reactive perpendicular aligned material, a liquid crystal display panel, and a method of liquid crystal alignment.

### BACKGROUND OF THE DISCLOSURE

With the development of the display technology, flat display devices such as a liquid crystal display (LCD) are widely employed in various consumer electronics like a mobile phone, a television, personal digital assistance, a digital camera, a laptop, a desktop computer, locating in the mainstream of display devices.

Most of liquid crystal display devices on the market are backlight liquid crystal displays, including liquid crystal display panels and backlight modules. The operational principle of a liquid crystal display panel is to dispose liquid crystal molecules on two parallel glass substrates, a number of vertical and horizontal weak currents are between the two glass substrates, which can control the liquid crystal molecules to redirect by power on/off, light from the backlight module can be refracted to show images.

A liquid crystal display panel can consist of a color filter (CF) substrate, a thin film transistor (TFT) substrate, a liquid crystal (LC) between the color filter substrate and the thin film transistor substrate, and a sealant.

A layer of thin film material is disposed on the CF substrate and the TFT substrate of a liquid crystal display respectively, mainly for aligning liquid crystal molecules, which is named an alignment film (mostly polyimide (PI) material). The main component of the alignment film is a friction alignment PI material or a light alignment PI material, however, both of which have respective flaws. First, the friction alignment PI material can cause problems like dust particles, static electricity residue, brushing streak, which can reduce yield of the product, though the light alignment PI material can avoid the problems above, heat resistance, ageing resistance and LC molecules anchorage are poor due to the property of material, affecting quality of a panel; second, PI materials have strong polarity and hygroscopicity, alignment can be inconsistent after storage and transportation, and the price of PI materials is high, a process of filming on a TFT-LCD is complicated, resulting in increasing costs of a panel. If alignment of liquid crystal molecules can be achieved without a PI film, costs of a panel can be reduced significantly.

### SUMMARY OF THE DISCLOSURE

The objective of the present disclosure is to provide a reactive perpendicular aligned material, a structure of which mainly includes three components—a head group, an intermediate group, and a tail group, primary intentions of the head group, on one hand, is to be anchored on a surface of an inorganic substrate utilizing its polar group by means of physical reaction, on the other hand, is to be anchored on a surface of a substrate by polymerization reaction of double bonds and reactive monomers; primary intentions of the intermediate group and the tail group are similar to that of a polyimide branch, vertically aligning liquid crystal molecules by stereo obstacle.

The present disclosure further provides a liquid crystal display panel, a liquid crystal layer includes liquid crystal molecules, the reactive perpendicular aligned materials above, and reactive monomers, under radiation of UV light, the reactive perpendicular aligned materials and the reactive monomers polymerize on a surface of a substrate to anchor liquid crystal molecules, not only simplifying the process of a TFT-LCD, costs of producing a TFT-LCD can also be reduced significantly.

The disclosure further provides a method for aligning liquid crystals, disposing liquid crystal molecules, reactive perpendicular aligned materials, and reactive monomers in a liquid crystal layer in a liquid crystal display panel, first applying voltage to the liquid crystal layer to redirect liquid crystal molecules, then radiating the liquid crystal layer with UV light, which can polymerize the reactive perpendicular aligned materials and the reactive monomers on surfaces of a first and a second substrates to anchor liquid crystal molecules, liquid crystal molecules engender a pre-tilt angle after releasing the voltage.

The disclosure provides a reactive perpendicular aligned material to achieve the purpose above with a general structural formula of A-Z—R, A is —CH═CH—COOH; Z is

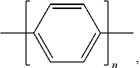

where n≧1; R represents a linear chained or a branched chained alkyl group with 5˜20 C atoms, a CH₂ group in the alkyl group is substituted by a phenyl group, a naphthenic base, —CONH—, —COO—, —O—CO—, —S—, —CO— or —CH═CH—, or a F atom or a Cl atom substitutes for a H atom in the alkyl group.

A structural formula of the reactive perpendicular aligned material is:

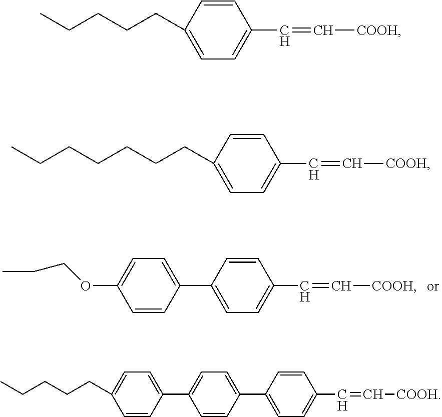 [SMILES: CCCCCc1ccc(C=CC(=O)O)cc1 ; CCCCCCCc1ccc(C=CC(=O)O)cc1 ; CCCOc1ccc(-c2ccc(C=CC(=O)O)cc2)cc1 ; CCCCCc1ccc(-c2ccc(-c3ccc(C=CC(=O)O)cc3)cc2)cc1]

The disclosure further provides a liquid crystal display panel, including a first substrate and a second substrate disposed opposite, a liquid crystal layer disposed between the first substrate and the second substrate, a first electrode disposed on a surface of the first substrate towards the liquid crystal layer, and a second electrode disposed on a surface of the second substrate towards the liquid crystal layer; the liquid crystal layer includes liquid crystal molecules, reactive perpendicular aligned materials, and reactive monomers polymerized with reactive perpendicular aligned materials under ultraviolet radiation; a general structural formula of the reactive perpendicular aligned material is A-Z—R, A is —CH═CH—COOH; Z is

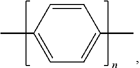

where n≧1; R represents a linear chained or a branched chained alkyl group with 5˜20 C atoms, a CH₂ group in the alkyl group is substituted by a phenyl group, a naphthenic base, —CONH—, —COO—, —O—CO—, —S—, —CO— or —CH═CH—, or a F atom or a Cl atom substitutes for a H atom in the alkyl group.

A structural formula of the reactive perpendicular aligned materials is:

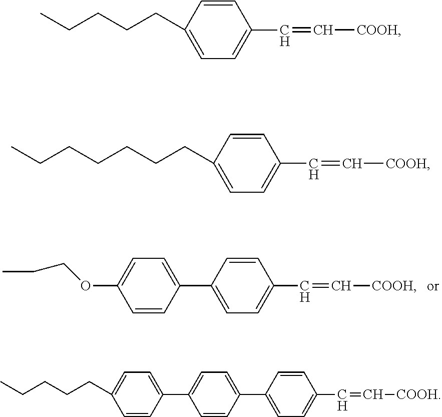 [SMILES: CCCCCc1ccc(C=CC(=O)O)cc1 ; CCCCCCCc1ccc(C=CC(=O)O)cc1 ; CCCOc1ccc(-c2ccc(C=CC(=O)O)cc2)cc1 ; CCCCCc1ccc(-c2ccc(-c3ccc(C=CC(=O)O)cc3)cc2)cc1]

The reactive monomer is one or more of following four compounds:

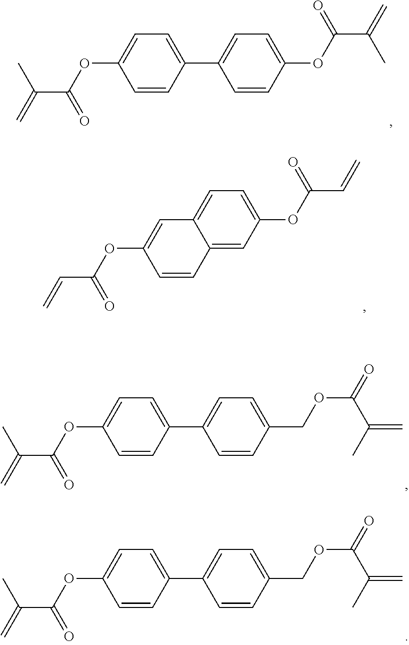

In the liquid crystal layer, content of the reactive perpendicular aligned materials is 0.1%˜5 wt %; content of the reactive monomer is 0.01˜0.1 wt %.

The first substrate and the second substrate are a CF substrate and a TFT substrate respectively; the first electrode and the second electrode are a common electrode and a pixel electrode respectively.

The disclosure further provides a method for aligning liquid crystals, including the following steps:

step 1, providing a liquid crystal display panel, including a first substrate and a second substrate disposed opposite, a liquid crystal layer disposed between the first substrate and the second substrate, a first electrode disposed on a surface of the first substrate towards the liquid crystal layer, and a second electrode disposed on a surface of the second substrate towards the liquid crystal layer; the liquid crystal layer including liquid crystal molecules, reactive perpendicular aligned materials, and reactive monomers that polymerize with reactive perpendicular aligned materials under ultraviolet radiation; a general structural formula of the reactive perpendicular aligned materials is A-Z—R, A is —CH═CH—COOH; Z is

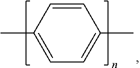

where n≧1; R represents a linear chained or a branched chained alkyl group with 5˜20 C atoms, a CH₂ group in the alkyl group is substituted by a phenyl group, a naphthenic base, —CONH—, —COO—, —O—CO—, —S—, —CO— or —CH═CH—, or a F atom or a Cl atom substitutes for a H atom in the alkyl group;

step 2, applying voltage to two sides of the liquid crystal layer by the first electrode and the second electrode to redirect liquid crystal molecules;

step 3, applying voltage to two sides of the liquid crystal layer and simultaneously radiating UV light on the liquid crystal display panel, polymerizing the reactive perpendicular aligned materials and the reactive monomers on the first and the second substrates to anchor liquid crystal molecules;

step 4, removing voltage from the two sides of the liquid crystal layer, making liquid crystal molecules to engender a pre-tilt angle.

In the liquid crystal layer in a liquid crystal display panel in the step 1, content of the reactive perpendicular aligned materials is 0.1%˜5 wt %; content of the reactive monomer is 0.01˜0.1 wt %.

In the step 2 and step 3, a value of the voltage applied to the two sides of the liquid crystal layer is 15˜25V; in the step 3, intensity of the UV light radiated on the liquid crystal display panel is 50˜85 mW/cm²; a wavelength of the UV light is 365 nm.

Advantages of the disclosure: the disclosure provides a reactive perpendicular aligned material, a liquid crystal display panel, and a method for aligning liquid crystals, a general structural formula of the reactive perpendicular aligned material is A-Z—R, a head group A plays a role in anchoring, on one hand, it can be anchored on a surface of a substrate utilizing the —COOH group by means of physical reaction, on the other hand, it can strengthen the ability of anchoring liquid crystal molecules by the reaction of a —CH═CH— group and a reactive monomer; primary intentions of an intermediate group Z and a tail group R are similar to that of a polyimide branch, vertically aligning liquid crystal molecules by stereo obstacle; a liquid crystal display panel adopting the reactive perpendicular aligned materials can be without an alignment film, which can simplify the process of a TFT-LCD as well as reducing costs of producing a TFT-LCD significantly; the method for aligning liquid crystals is easily processed and effective in alignment.

In order to more clearly illustrate the embodiments of the present disclosure, detailed description and figures relevant to the disclosure can be referred, it is obvious that the drawings are merely for reference and embodiments instead of limiting the disclosure.

### BRIEF DESCRIPTION OF THE DRAWINGS

Embodiments of the disclosure will be described in detail and illustrated by figures to clarify the proposal and the advantages of the disclosure.

In figures,

FIG. 1 is a structural diagram of a reactive perpendicular aligned material of the present disclosure;

FIG. 2 is a structural diagram of a liquid crystal display panel, which is also a diagram of step 1 of a method for aligning liquid crystals of the disclosure;

FIG. 3 is a diagram of step 2 of a method for aligning liquid crystals of the disclosure;

FIG. 4 is a diagram of step 3 of a method for aligning liquid crystals of the disclosure;

FIG. 5 is a diagram of step 4 of a method for aligning liquid crystals of the disclosure;

FIG. 6 is a scanning electron microscope photograph of a polymer layer obtained by polymerization of reactive perpendicular aligned materials and reactive monomers on a surface of a substrate in step 3 of a method for aligning liquid crystals according to a preferred embodiment of the disclosure.

### DETAILED DESCRIPTION OF PREFERRED EMBODIMENTS

Embodiments of the present disclosure are described in detail with the accompanying drawings to illustrate the proposal and performance as follows.

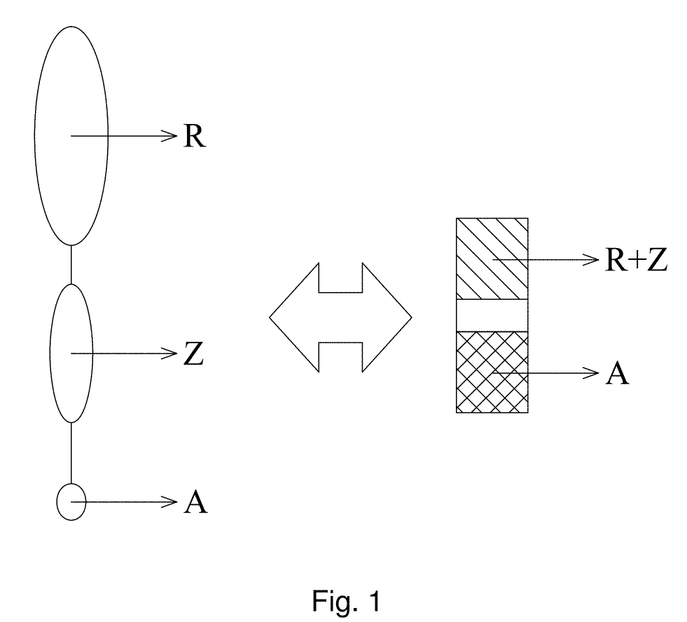

Referring to FIG. 1, The disclosure provides a reactive perpendicular aligned material with a general structural formula of A-Z—R, A is —CH═CH—COOH; Z is

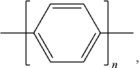

where n≧1; R represents a linear chained or a branched chained alkyl group with 5˜20 C atoms, a CH₂ group in the alkyl group is substituted by a phenyl group, a naphthenic base, —CONH—, —COO—, —O—CO—, —S—, —CO— or —CH═CH—, or a F atom or a Cl atom substitutes for a H atom in the alkyl group.

Preferably, a structural formula of the reactive perpendicular aligned material is:

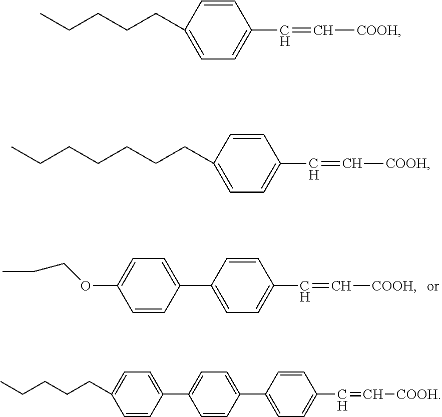 [SMILES: CCCCCc1ccc(C=CC(=O)O)cc1 ; CCCCCCCc1ccc(C=CC(=O)O)cc1 ; CCCOc1ccc(-c2ccc(C=CC(=O)O)cc2)cc1 ; CCCCCc1ccc(-c2ccc(-c3ccc(C=CC(=O)O)cc3)cc2)cc1]

The disclosure provides a reactive perpendicular aligned material with a general structural formula of A-Z—R, a head group A plays a role in anchoring, on one hand, it can be anchored on a surface of a substrate utilizing the —COOH group by means of physical reaction, on the other hand, it can strengthen the ability of anchoring liquid crystal molecules by the reaction of a —CH═CH— group and a reactive monomer; primary intentions of an intermediate group Z and a tail group R are similar to that of a polyimide branch, vertically aligning liquid crystal molecules by stereo obstacle; a liquid crystal display panel adopting the reactive perpendicular aligned materials can be without an alignment film, which can simplify the process of a TFT-LCD as well as reducing costs of producing a TFT-LCD significantly.

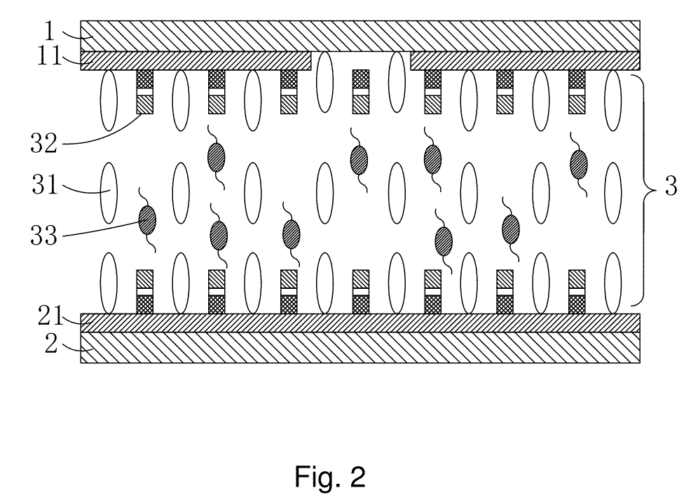

Referring to FIG. 2, the disclosure further provides a liquid crystal display panel, including a first substrate 1 and a second substrate 2 disposed opposite, a liquid crystal layer 3 disposed between the first substrate 1 and the second substrate 2, a first electrode 11 disposed on a surface of the first substrate 1 towards the liquid crystal layer 3, and a second electrode 21 disposed on a surface of the second substrate 2 towards the liquid crystal layer 3; the liquid crystal layer 3 includes liquid crystal molecules 31, reactive perpendicular aligned materials 32, and reactive monomers 33 polymerized with reactive perpendicular aligned materials 32 under ultraviolet radiation; a general structural formula of the reactive perpendicular aligned materials 32 is A-Z—R, A is —CH═CH—COOH; Z is

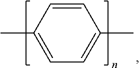

where n≧1; R represents a linear chained or a branched chained alkyl group with 5˜20 C atoms, a CH₂ group in the alkyl group is substituted by a phenyl group, a naphthenic base, —CONH—, —COO—, —O—CO—, —S—, —CO— or —CH═CH—, or a F atom or a Cl atom substitutes for a H atom in the alkyl group.

Preferably, a structural formula of the reactive perpendicular aligned materials 32 is:

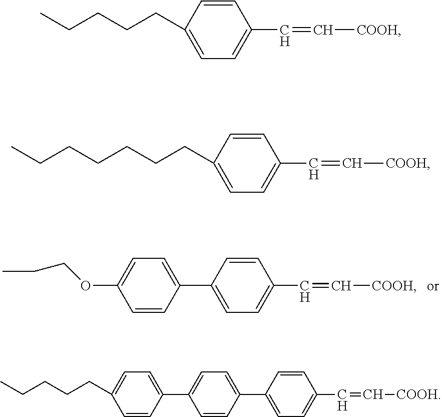 [SMILES: CCCCCc1ccc(C=CC(=O)O)cc1 ; CCCCCCCc1ccc(C=CC(=O)O)cc1 ; CCCOc1ccc(-c2ccc(C=CC(=O)O)cc2)cc1 ; CCCCCc1ccc(-c2ccc(-c3ccc(C=CC(=O)O)cc3)cc2)cc1]

Specifically, the reactive monomer 33 can be any reactive monomer in polymer stabilized vertically aligned (PSVA) technology.

Preferably, the reactive monomer 33 can be one or more of following four compounds:

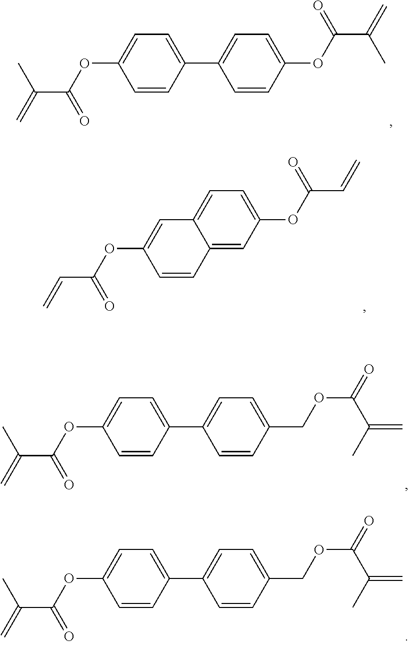

Preferably, in the liquid crystal layer 3, content of the reactive perpendicular aligned materials 32 is 0.1%˜5 wt %; content of the reactive monomers 33 is 0.01˜0.1 wt %.

Specifically, the first substrate 1 and the second substrate 2 are a CF substrate and a TFT substrate respectively; the first electrode 11 and the second electrode 21 are a common electrode and a pixel electrode respectively.

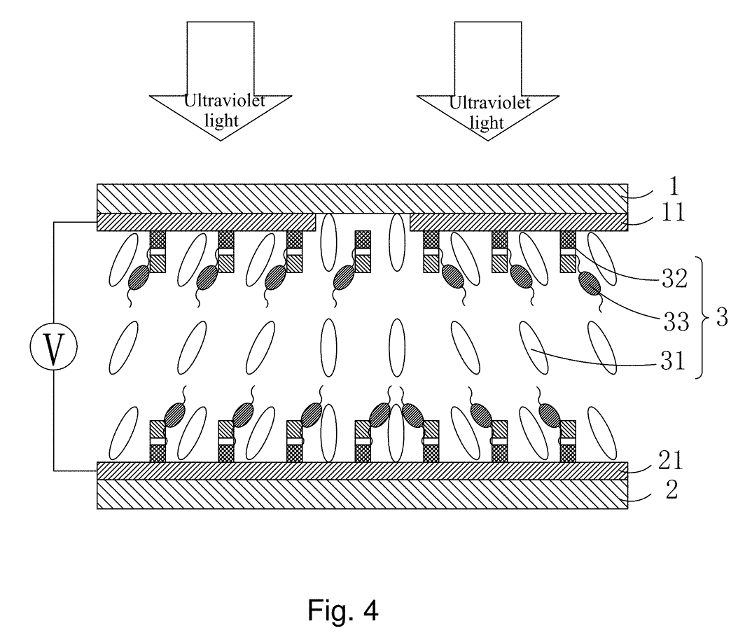

Referring to FIGS. 2-5, the disclosure further provides a method for aligning liquid crystals, including the following steps:

step 1, as shown in FIG. 2, providing a liquid crystal display panel, including a first substrate 1 and a second substrate 2 disposed opposite, a liquid crystal layer 3 disposed between the first substrate 1 and the second substrate 2, a first electrode 11 disposed on a surface of the first substrate 1 towards the liquid crystal layer 3, and a second electrode 21 disposed on a surface of the second substrate 2 towards the liquid crystal layer 3; the liquid crystal layer 3 including liquid crystal molecules 31, reactive perpendicular aligned materials 32, and reactive monomers 33 polymerized with the reactive perpendicular aligned materials 32 under ultraviolet radiation; the liquid crystal molecules are arranged perpendicularly to the first substrate 1 and the second substrate 2.

Specifically, a general structural formula of the reactive perpendicular aligned materials 32 is A-Z—R, A is —CH═CH—COOH; Z is

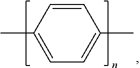

where n≧1; R represents a linear chained or a branched chained alkyl group with 5˜20 C atoms, a CH₂ group in the alkyl group is substituted by a phenyl group, a naphthenic base, —CONH—, —COO—, —O—CO—, —S—, —CO— or —CH═CH—, or a F atom or a Cl atom substitutes for a H atom in the alkyl group.

Preferably, a structural formula of the reactive perpendicular aligned materials 32 is:

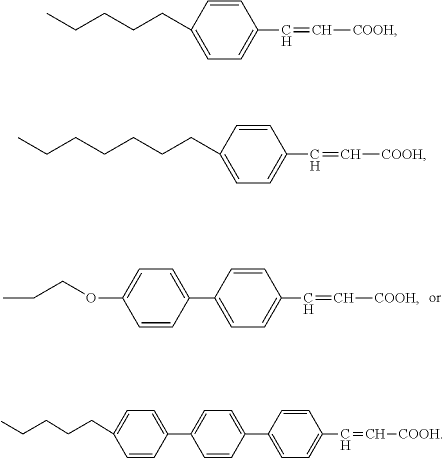 [SMILES: CCCCCc1ccc(C=CC(=O)O)cc1 ; CCCCCCCc1ccc(C=CC(=O)O)cc1 ; CCCOc1ccc(-c2ccc(C=CC(=O)O)cc2)cc1 ; CCCCCc1ccc(-c2ccc(-c3ccc(C=CC(=O)O)cc3)cc2)cc1]

Specifically, the reactive monomer 33 can be any reactive monomer in polymer stabilized vertically aligned (PSVA) technology.

Preferably, the reactive monomer 33 can be one or more of following four compounds:

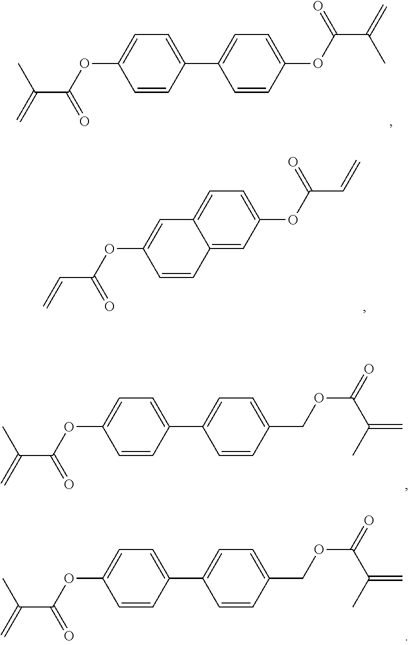

Preferably, in the liquid crystal layer 3, content of the reactive perpendicular aligned materials 32 is 0.1%˜5 wt %; content of the reactive monomers 33 is 0.01˜0.1 wt %.

Specifically, the first substrate 1 and the second substrate 2 are a CF substrate and a TFT substrate respectively; the first electrode 11 and the second electrode 21 are a common electrode and a pixel electrode respectively.

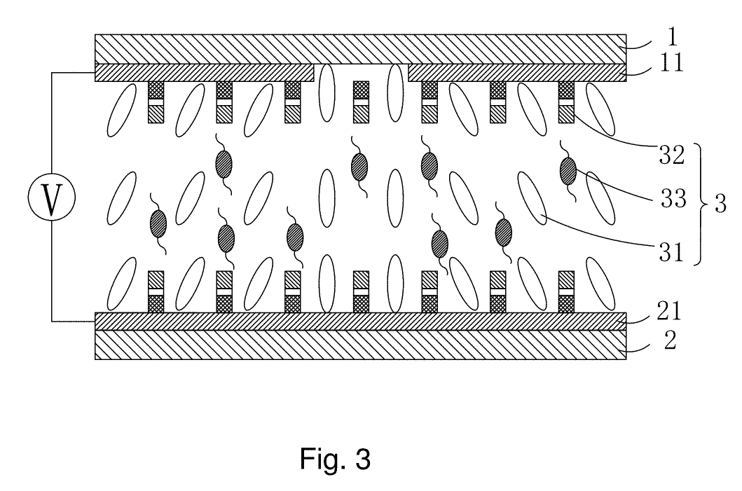

Step 2, as shown in FIG. 3, applying a voltage of 15˜25V to two sides of the liquid crystal layer 3 by the first electrode 11 and the second electrode 21 to redirect liquid crystal molecules 31.

Step 3, applying the voltage of 15˜25V to two sides of the liquid crystal layer 3 and simultaneously radiating UV light with intensity of 50˜85 mW/cm² on the liquid crystal display panel, polymerizing the reactive perpendicular aligned materials 32 and the reactive monomers 33 on the first substrate 1 and the second substrate 2 to anchor liquid crystal molecules 31.

Preferably, a wavelength of the UV light is 365 nm.

Specifically, in the step 3, the polymerization reaction of the reactive perpendicular aligned materials 32 and the reactive monomers 33 is a sort of free radical polymerization reaction: under radiation of UV light, double bonds in the reactive monomers break to generate free radicals, which can lead to a polymerization reaction, the reactive monomers 33 are self-polymerizing and polymerizing with double bonds in the reactive perpendicular aligned materials 32 simultaneously, which can form a crosslinked net structure to intensify ability of anchoring liquid crystal molecules.

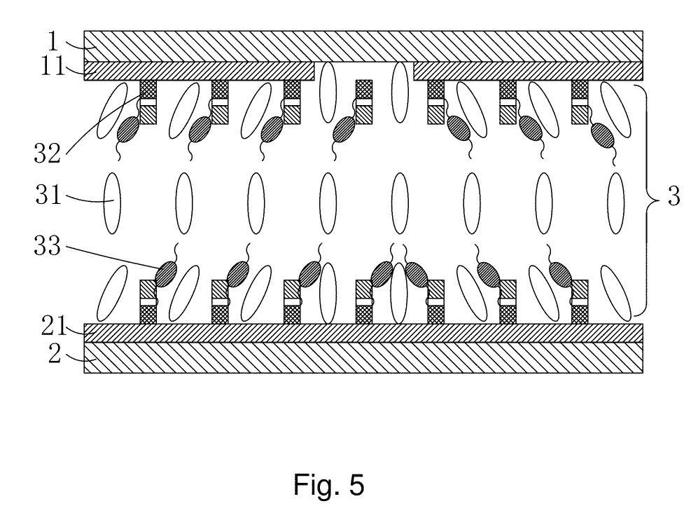

Step 4, as shown in FIG. 5, removing voltage from the two sides of the liquid crystal layer 3, making liquid crystal molecules 31 to engender a pre-tilt angle.

In a preferred embodiment of a method for aligning liquid crystals of the disclosure, a reactive perpendicular aligned material in a liquid crystal layer of a liquid crystal display panel provided by step 1 is

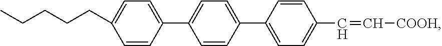 [SMILES: CCCCCc1ccc(-c2ccc(-c3ccc(C=CC(=O)O)cc3)cc2)cc1]

a reactive monomer is

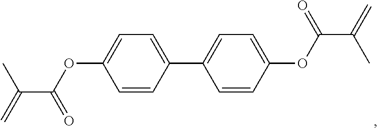 [SMILES: C=C(C)C(=O)Oc1ccc(-c2ccc(OC(=O)C(=C)C)cc2)cc1]

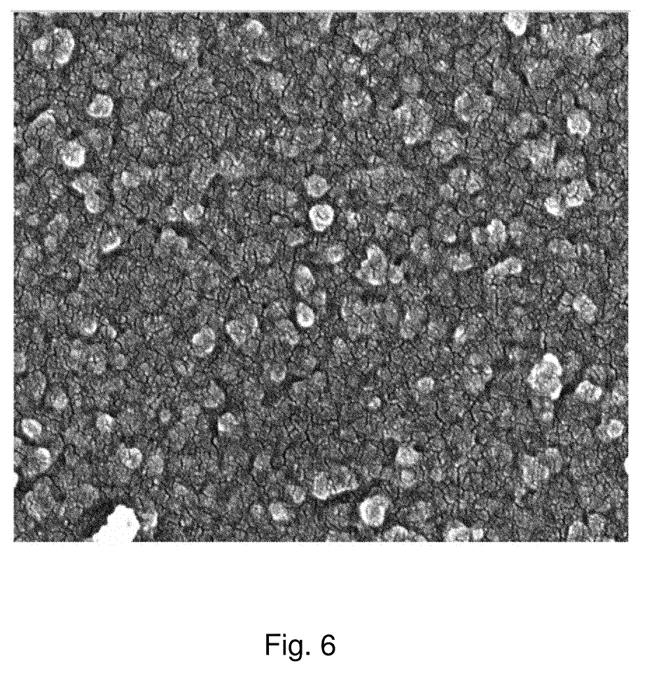

after step 3, reactive perpendicular aligned materials and reactive monomers polymerize on surfaces of a first and a second substrates to form a polymer layer, a photograph of the polymer layer amplified by a scanning electron microscope with 160-hundred-fold is shown in FIG. 6. And after step 4, an achieved liquid crystal display panel is dim under a condition of power-off, which can prove effects of liquid crystal vertical alignment with the method of aligning liquid crystals utilizing reactive perpendicular aligned materials.

The present disclosure provides a liquid crystal display panel, a liquid crystal layer includes liquid crystal molecules 31, the reactive perpendicular aligned materials 32, and reactive monomers 33 that polymerize with the reactive perpendicular aligned materials 32 under radiation of UV light, a general structural formula of the reactive perpendicular aligned materials 32 is A-Z—R, a head group A plays a role in anchoring, on one hand, it can be anchored on a surface of a substrate utilizing the —COOH group by means of physical reaction, on the other hand, it can strengthen the ability of anchoring liquid crystal molecules by the reaction of a —CH═CH— group and a reactive monomer; primary intentions of an intermediate group Z and a tail group R are similar to that of a polyimide branch, vertically aligning liquid crystal molecules by stereo obstacle; a liquid crystal display panel adopting the reactive perpendicular aligned materials can be without an alignment film, which can simplify the process of a TFT-LCD as well as reducing costs of producing a TFT-LCD significantly.

Embodiments I and II are employed to describe preparation methods of two specific structural reactive perpendicular aligned materials:

### Embodiment I

A preparation method of a compound

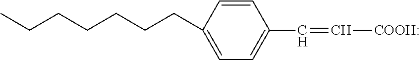

step 1, measuring aromatic amines (I), HCl, NaNO₂ with a mole ratio of 1:(1˜5):(1.01˜1.10), placing the measured aromatic amines (I), hydrochloric acid, and NaNO₂ in a reactor and stirring at 0˜5° C. for 3˜5 hours, adding KI with a mole ratio of 1˜1.2 compared with aromatic amines (I), a product iodobenzene (II) produced after reaction at 25° C. for 1˜5 hours; a reaction equation of the step 1 is as follows:

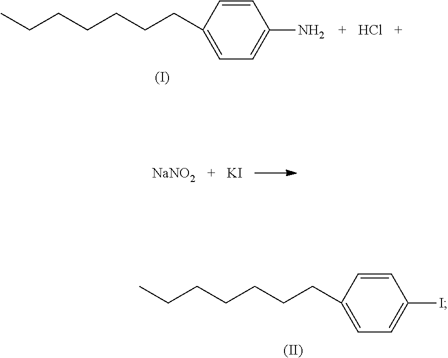

step 2, measuring iodobenzene (II) produced in the step 1 above and acrylic acid with a mole ratio of 1:(2˜3), dissolving the measured iodobenzene (II) and acrylic acid in solvent, employing metal palladium as a catalyst, a compound (III) prepared after reaction at 100° C. for 15˜25 hours; specifically, in the step 2, the solvent can be N-methyl pyrrolidone.

A reaction equation of the step 2 is:

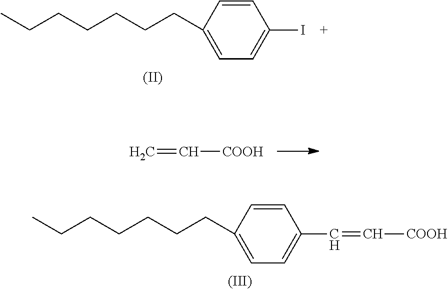

The produced compound (III) is analyzed by nuclear magnetic resonance, nuclear magnetic resonance data obtained is: δ=0.96 (3H), δ=1.33 (2H), δ=1.29 (2H), δ=1.62 (2H), δ=2.55 (2H), δ=7.18 (2H), δ=7.43 (2H), δ=7.54 (4H), 7.43 (2H), δ=7.36 (2H), δ=7.61 (1H), δ=6.41 (1H), δ=11.0 (1H), which can determine a structural formula of the compound (III) is

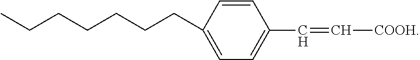 [SMILES: CCCCCCCc1ccc(C=CC(=O)O)cc1]

### Embodiment II

A preparation method of a compound

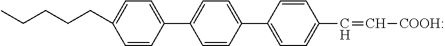

step 1, measuring aromatic nitrile (IV) and LiAlH₄ with a mole ratio of 1:(1˜3), measuring tetrahydrofuran (THF) with a ratio of 3:1 in mol:L compared with aromatic nitrile (IV), dissolving the measured aromatic nitrile (IV) and LiAlH₄ in tetrahydrofuran; reacting at 72° C. by heating and refluxing for 1˜5 hours, then adding ice water solution including 20 wt % sodium hydroxide with a volume ratio of V(NaOH)/V(THF)=2/1 in the reaction, aromatic nitrile (IV) prepared; a reaction equation of the step 1 is as follows:

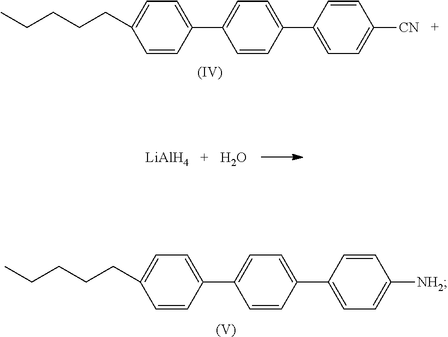

step 2, measuring aromatic nitrile (IV) produced in the step 1 above, HCl and NaNO₂ with a mole ratio of 1:(1˜5):(1.01:1.10), placing the measured aromatic nitrile (IV), hydrochloric acid and NaNO₂ in a reactor, stirring at 0˜5° C. for 3˜5 hours, adding KI with a mole ratio of 1˜1.2 compared with aromatic nitrile (IV), a product aromatic nitrile (IV) produced after reaction at 25° C. for 1˜5 hours; a reaction equation of the step 2 is as follows:

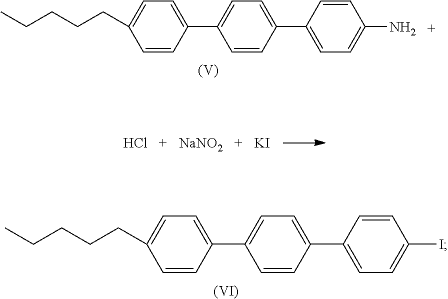

step 3, measuring aromatic nitrile (IV) produced in the step 2 above and acrylic acid with a mole ratio of 1:(2˜3), dissolving the measured aromatic nitrile (IV) and acrylic acid in solvent, employing metal palladium as a catalyst, a compound (VII) prepared after reaction at 100° C. for 15˜25 hours; specifically, in the step 3, the solvent can be N-methyl pyrrolidone.

A reaction equation of the step 3 is:

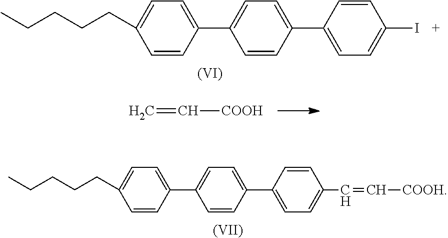

The produced compound (VII) is analyzed by nuclear magnetic resonance, nuclear magnetic resonance data obtained is: δ=0.96 (3H), δ=1.33 (2H), δ=1.29 (2H), δ=1.62 (2H), δ=2.55 (2H), δ=7.18 (2H), δ=7.43 (2H), δ=7.54 (4H), δ=7.43 (2H), δ=7.36 (2H), δ=7.61 (1H), δ=6.41 (1H), δ=11.0 (1H), which can determine a structural formula of the compound (VII) is

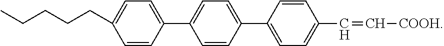 [SMILES: CCCCCc1ccc(-c2ccc(-c3ccc(C=CC(=O)O)cc3)cc2)cc1]

Overall, the disclosure provides a reactive perpendicular aligned material, a liquid crystal display panel, and a method for aligning liquid crystals, a general structural formula of the reactive perpendicular aligned material is A-Z—R, a head group A plays a role in anchoring, on one hand, it can be anchored on a surface of a substrate utilizing the —COOH group by means of physical reaction, on the other hand, it can strengthen the ability of anchoring liquid crystal molecules by the reaction of a —CH═CH— group and a reactive monomer; primary intentions of an intermediate group Z and a tail group R are similar to that of a polyimide branch, vertically aligning liquid crystal molecules by stereo obstacle; a liquid crystal display panel adopting the reactive perpendicular aligned material can be without an alignment film, which can simplify the process of a TFT-LCD as well as reducing costs of producing a TFT-LCD significantly; the method for aligning liquid crystals is easily processed and effective in alignment.

It is understandable in practical to a person skilled in the art that all or portion of the processes in the method according to the aforesaid embodiment can be accomplished with modifications and equivalent replacements, which should be covered by the protected scope of the disclosure.

What is claimed is:

1. A reactive perpendicular aligned material, whose general structural formula is A-Z—R, wherein A is —CH═CH—COOH;
Z is 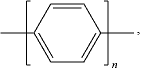
wherein ≧1;
R represents a linear chained or a branched chained alkyl group with 5˜20 C atoms, a CH₂ group in the alkyl group is substituted by a phenyl group, a naphthenic base, —CONH—, —COO—, —O—CO—, —S—, —CO— or —CH═CH—, or aF atom or a Cl atom substitutes for a H atom in the alkyl group.

2. The reactive perpendicular aligned material according to claim 1, wherein a structural formula of the reactive perpendicular aligned material is:

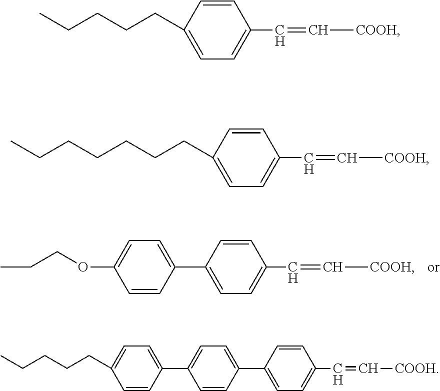 [SMILES: CCCCCc1ccc(C=CC(=O)O)cc1 ; CCCCCCCc1ccc(C=CC(=O)O)cc1 ; CCCOc1ccc(-c2ccc(C=CC(=O)O)cc2)cc1 ; CCCCCc1ccc(-c2ccc(-c3ccc(C=CC(=O)O)cc3)cc2)cc1]

3. A liquid crystal display panel, comprising a first substrate and a second substrate disposed opposite, a liquid crystal layer disposed between the first substrate and the second substrate, a first electrode disposed on a surface of the first substrate towards the liquid crystal layer, and a second electrode disposed on a surface of the second substrate towards the liquid crystal layer; the liquid crystal layer comprising liquid crystal molecules, reactive perpendicular aligned materials, and reactive monomers that polymerize with a reactive perpendicular aligned material under ultraviolet radiation;
a general structural formula of the reactive perpendicular aligned material is A-Z—R, wherein A is —CH═CH—COOH; Z is 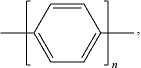
where n≧1; R represents a linear chained or a branched chained alkyl group with 5˜20 C atoms, a CH₂ group in the alkyl group is substituted by a phenyl group, a naphthenic base, —CONH—, —COO—, —O—CO—, —S—, —CO— or —CH═CH—, or a F atom or a Cl atom substitutes for a H atom in the alkyl group.

4. The liquid crystal display panel according to claim 3, wherein a structural formula of the reactive perpendicular aligned material is:

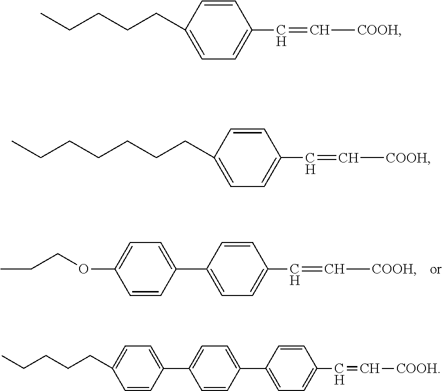 [SMILES: CCCCCc1ccc(C=CC(=O)O)cc1 ; CCCCCCCc1ccc(C=CC(=O)O)cc1 ; CCCOc1ccc(-c2ccc(C=CC(=O)O)cc2)cc1 ; CCCCCc1ccc(-c2ccc(-c3ccc(C=CC(=O)O)cc3)cc2)cc1]

5. The liquid crystal display panel according to claim 3, wherein the reactive monomer is one or more of following four compounds:

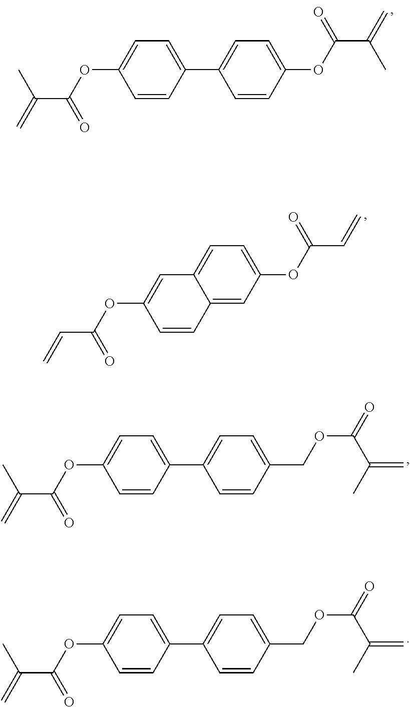

6. A liquid crystal display panel according to claim 3, wherein in the liquid crystal layer, content of the reactive perpendicular aligned materials is 0.1%˜5 wt %; content of the reactive monomer is 0.01˜0.1 wt %.

7. The liquid crystal display panel according to claim 3, wherein the first substrate and the second substrate are a CF substrate and a TFT substrate respectively; the first electrode and the second electrode are a common electrode and a pixel electrode respectively.

8. A method for aligning liquid crystals, comprising the following steps:
step 1, providing a liquid crystal display panel, comprising a first substrate and a second substrate disposed opposite, a liquid crystal layer disposed between the first substrate and the second substrate, a first electrode disposed on a surface of the first substrate towards the liquid crystal layer, and a second electrode disposed on a surface of the second substrate towards the liquid crystal layer; the liquid crystal layer comprising liquid crystal molecules, reactive perpendicular aligned materials, and reactive monomers that polymerize with a reactive perpendicular aligned material under ultraviolet radiation;
a general structural formula of the reactive perpendicular aligned material being A-Z—R, wherein A is —CH═CH—COOH; Z is 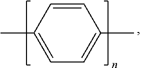
where n≧1; R represents a linear chained or a branched chained alkyl group with 5˜20 C atoms, a CH₂ group in the alkyl group is substituted by a phenyl group, a naphthenic base, —CONH—, —COO—, —O—CO—, —S—, —CO— or —CH═CH—, or a F atom or a Cl atom substitutes for a H atom in the alkyl group;
step 2, applying voltage to two sides of the liquid crystal layer by the first electrode and the second electrode, redirecting liquid crystal molecules;
step 3, applying voltage to two sides of the liquid crystal layer and simultaneously radiating UV light on a liquid crystal display panel, polymerizing the reactive perpendicular aligned materials and the reactive monomers on the first and the second substrates to anchor liquid crystal molecules;
step 4, removing voltage from the two sides of the liquid crystal layer, making liquid crystal molecules to engender a pre-tilt angle.

9. The method for aligning liquid crystals according to claim 8, wherein in the liquid crystal layer in the liquid crystal display panel in the step 1, content of the reactive perpendicular aligned materials is 0.1%˜5 wt %; content of the reactive monomer is 0.01˜0.1 wt %.

10. The method for aligning liquid crystals according to claim 8, wherein in the step 2 and step 3, a value of the voltage applied to the two sides of the liquid crystal layer is 15˜25V; in the step 3, intensity of the UV light radiated on the liquid crystal display panel is 50˜85 mW/cm²; a wavelength of the UV light is 365 nm.
